(19) United States
(12) Patent Application Publication
(10) Pub. No.: US 2023/0021405 A1
(43) Pub. Date: Jan. 26, 2023

(54) CLOSURE DEVICES AND METHODS FOR SEALING BIOLOGIC TISSUE MEMBRANES

(71) Applicant: Deinde Medical Corp., Miramar, FL (US)

(72) Inventor: Robert C. Farnan, Fort Lauderdale, FL (US)

(21) Appl. No.: 17/959,834

(22) Filed: Oct. 4, 2022

Related U.S. Application Data
(63) Continuation of application No. PCT/US21/19158, filed on Feb. 23, 2021.
(60) Provisional application No. 63/009,781, filed on Apr. 14, 2020.

(51) Int. Cl. A61B 17/00

(52) U.S. Cl. CPC A61B 17/0057; A61B 2017/00676; A61B 2017/00898

(57) ABSTRACT

A device for sealing an opening through a biologic tissue membrane against leakage. The device includes an implantable fluid sealing plug including a structural hydrogel. The fluid sealing plug is configured to be positioned at least partially within the opening through a biologic tissue such as a membrane. The fluid sealing plug is configured to increase in diameter upon absorbing a fluid, and the increase in diameter of the fluid sealing plug upon absorbing the fluid seals the opening through a biologic tissue membrane.

### CROSS REFERENCE TO RELATED APPLICATION

This application is a continuation of PCT Application Serial No. PCT/US2021/019158, filed Feb. 23, 2021 which claims the priority of U.S. Provisional Patent Application Ser. No. 63/009,781 filed on Apr. 14, 2020, the disclosures of which are incorporated herein by reference in their entirety.

### TECHNICAL FIELD

The present invention generally relates to implantable tissue closure devices and, more particularly, to implantable closure devices for sealing punctures or other openings through biologic tissue membranes, such as the meninges, against leakage of biological fluids, such as cerebrospinal fluid, and related methods.

### BACKGROUND

The present disclosure contemplates that the meninges are protective biologic tissue membranes around the brain and spinal cord. The meninges contain the cerebrospinal fluid and generally form a conduit that surrounds the spinal cord and the cerebral ventricles. In some medical or surgical procedures, a needle or other instrument may be used to puncture through the skin, soft tissue, and the meninges, such as to gain access to the cerebrospinal fluid. When the instrument is removed, the hole or puncture may not seal spontaneously, such as due to the inelastic properties of the meninges. If the puncture does not promptly seal, the cerebrospinal fluid may leak into the adjacent soft tissue, which may not be clinically desirable. In other medical situations an opening may exist through tissue, such as the result a natural tissue opening or a surgically formed tissue opening, an injury, etc.

Accordingly, and in spite of the various advances already made in this field, there is a need for further improvements related to implantable tissue closure devices and, more particularly, to implantable closure devices for sealing openings, such as punctures through biologic tissue membranes, such as the meninges, and related methods.

### SUMMARY

Generally, a device is provided for sealing an opening through a biologic tissue membrane against leakage. The device includes an elongated support element comprising a support element distal portion and a support element proximal portion. The device further includes an implantable fluid sealing plug disposed on the support element distal portion. The fluid sealing plug comprises a structural hydrogel and is configured to increase in diameter upon absorbing a fluid. The support element with the fluid sealing plug disposed thereon is configured to be positioned at least partially within the opening through a biologic tissue membrane. The device may include various other features, some of which are discussed herein.

The fluid sealing plug may be generally elongated and is disposed along the support element distal portion. The fluid sealing plug may be disposed substantially coaxially circumferentially around the support element distal portion. The structural hydrogel may be configured such that, when fully hydrated, the fluid sealing plug is generally in the form of an elongated generally cylindrical element, or even more specifically, a right circular cylinder. When fully hydrated, a fluid sealing plug diameter may be substantially constant over a fluid sealing plug length. When substantially dehydrated, a fluid sealing plug diameter may be substantially constant over a fluid sealing plug length. The support element distal portion may extend distally beyond a distal end portion of the fluid sealing plug. The structural hydrogel may be substantially dehydrated until use. The fluid sealing plug may be configured such that, prior to implantation in the opening through the biologic tissue membrane, the structural hydrogel is at least partially hydrated by the fluid. When fully hydrated, the structural hydrogel may comprise about 5% to about 10% solids. The structural hydrogel may comprise at least one of amorphous thermoplastic urethane and hydrolyzed polyacrylicnitrile. The support element may be substantially rigid or semi-rigid and my comprise a metal wire. The metal wire may comprise at least one of nickel titanium, stainless steel, tungsten, and platinum. Alternatively, the support element may comprise a plastic rod. As another optional feature, the support element may be substantially flexible. The support element may comprise at least one filament, such as a suture material and the suture material may be bioabsorbable.

The device may further include an elongated, tubular pusher sleeve slidably disposed circumferentially around and along at least a portion of the support element proximal portion. The pusher sleeve includes a pusher sleeve length; and the pusher sleeve length may be less than a support element proximal portion length. A pusher sleeve distal end portion may be disposed in abutting relation to a fluid sealing plug proximal end portion. An axial stop may be releasably engageable with the support element proximal portion to selectively oppose proximal movement of the pusher sleeve relative to the support element. The axial stop may be configured to selectively oppose proximal movement of the pusher sleeve relative to the support element by selectively abutting a pusher sleeve proximal end portion. The axial stop may be configured to selectively hold a pusher sleeve distal end portion in abutting relation with a proximal end portion of the fluid sealing plug. The axial stop may be selectively axially slidable on the support element proximal portion. The axial stop may comprise an engagement mechanism configured to selectively releasably engage the support element proximal portion. The engagement mechanism may comprise a spring and a slide. The device may further comprise a hydration vial configured to receive therein the fluid and the support element distal portion with the fluid sealing plug disposed thereon. The hydration vial may comprise an interior cavity that is generally in the form of a cylinder, such as a right circular cylinder. The interior cavity may have an interior length that is longer than a fluid sealing plug length. The interior cavity may have an interior diameter that is greater than a fluid sealing plug diameter when the fluid sealing plug is fully hydrated.

The device may further comprise a sheath configured to deliver the fluid sealing plug to the opening through the biologic tissue membrane. The sheath may be configured to receive the fluid sealing plug therethrough; and the sheath may be configured to receive at least a portion of the support element therein. The device may further comprise an elongated, tubular pusher sleeve slidably disposed circumferentially around and along at least a portion of the support element proximal portion; and the sheath may be configured to receive at least a portion of the pusher sleeve therein. The sheath may be configured to receive the pusher sleeve entirely therein.

In another aspect, the invention provides a method of closing an opening through a biologic tissue membrane and sealing against leakage. The method comprises inserting an implantable fluid sealing plug disposed on an elongated support element into the opening through a biologic tissue membrane, the fluid sealing plug comprising a structural hydrogel.

The method may include various optional features and/or methodology. Prior to inserting the fluid sealing plug, the fluid sealing plug may be at least partially hydrated with a fluid. At least partially hydrating the fluid sealing plug may comprise increasing the fluid sealing plug diameter by absorbing the fluid into the fluid sealing plug. At least partially hydrating the fluid sealing plug may comprise substantially fully hydrating the fluid sealing plug. The fluid may comprise at least one of water, saline solution, blood, amniotic fluid, cerebrospinal fluid, polyethylene glycol, a thrombolytic agent, and a contrast agent. At least partially hydrating the fluid sealing plug may comprise hydrating a first portion of the fluid sealing plug with a first fluid and hydrating a second portion of the fluid sealing plug with a second fluid, the second fluid being different than the first fluid. The first fluid may comprise cerebrospinal fluid; and the second fluid may comprise blood. Prior to inserting the fluid sealing plug, the method may include positioning a sheath to deliver the fluid sealing plug through the sheath and into the opening through the biologic tissue membrane; and inserting the fluid sealing plug may comprise inserting the fluid sealing plug and the support element into the sheath. Inserting the fluid sealing plug may comprise inserting a pusher sleeve into the sheath, the support element extending through the pusher sleeve, and the fluid sealing plug being disposed on the support element distal to the pusher sleeve. After inserting the fluid sealing plug, the method may include withdrawing the sheath proximally off of the fluid sealing plug, the support element, and the pusher sleeve. Inserting the fluid sealing plug may comprise inserting the support element, the fluid sealing plug, and the pusher sleeve into the sheath until a stop disposed on the support element in abutting relation to a pusher sleeve proximal end portion abuts a proximal end of the sheath. The method may further comprise after inserting the fluid sealing plug, removing the stop from the support element; and withdrawing the sheath proximally off of the fluid sealing plug, the support element, and the pusher sleeve. After withdrawing the sheath, the method may further include detaching the support element from the fluid sealing plug and withdrawing the support element proximally through the pusher sleeve. After withdrawing the support element, the pusher sleeve may be withdrawn. After withdrawing the sheath, the pusher sleeve may be withdrawn proximally. After withdrawing the pusher sleeve, the support element may be cut while the support element remains attached to the fluid sealing plug. Cutting the support element may comprise cutting the support element near a skin surface of the patient. After inserting the implantable fluid sealing plug disposed on the elongated support element, the method may further comprise removing the fluid sealing plug and the elongated support element from the opening through the biologic tissue membrane. After inserting the implantable fluid sealing plug disposed on the elongated support element, the method may include repositioning the fluid sealing plug disposed and the elongated support element within the opening through the biologic tissue membrane.

In another aspect, the invention provides a device for sealing an opening through a biologic tissue membrane against leakage, including: an implantable fluid sealing plug comprising a structural hydrogel, the fluid sealing plug being configured to increase in diameter upon absorbing a fluid. The fluid sealing plug is configured to be positioned at least partially within the opening through a biologic tissue membrane. The device may have various optional features, such as, but not limited to, any of the features discussed herein.

In another aspect, the invention more generally provides a method of closing an opening through a biologic tissue membrane and sealing against leakage, the method comprising implanting a fluid sealing plug in the opening through a biologic tissue membrane, the fluid sealing plug comprising a structural hydrogel. The method may include various optional features or methodology such as, but not limited to, such features and/or methodology discussed herein.

The invention, in another aspect, provides a method of manufacturing a structural hydrogel biologic tissue closure device. This method generally includes disposing an implantable, elongated fluid sealing plug on a support element distal portion of an elongated support element, the fluid sealing plug comprising a structural hydrogel. The fluid sealing plug is configured to increase in diameter upon absorbing a fluid. The fluid sealing plug is configured to be positioned at least partially within an opening through a biologic tissue membrane.

The manufacturing method may include additional and/or optional features and/or methodology. For example, disposing the fluid sealing plug on the support element may comprise disposing the fluid sealing plug substantially coaxially circumferentially around and along the support element distal portion. Disposing the fluid sealing plug on the support element may comprise disposing the fluid sealing plug on the support element so that the support element distal portion extends distally beyond a distal end portion of the fluid sealing plug. The method may further comprise installing a retainer on the support element distal portion distal to the distal end portion of the fluid sealing plug. The support element may comprise a flexible filament; and installing the retainer may comprise tying a knot in the filament. The fluid sealing plug includes a fluid sealing plug diameter that may be substantially constant over a fluid sealing plug length. The fluid sealing plug may be substantially dehydrated. The method may further comprise inserting a support element proximal portion into an elongated, tubular pusher sleeve. The method may further comprise abutting a pusher sleeve distal end portion against a fluid sealing plug proximal end portion. The method may further comprise installing a removable stop on the support element proximal portion proximal to the pusher sleeve. The method may further comprise abutting the stop against a pusher sleeve proximal end portion.

In yet another aspect, the invention provides a device for sealing an opening through a biologic tissue membrane against leakage. The device comprises an elongated support element comprising a support element distal portion and a support element proximal portion; and an implantable fluid sealing plug disposed on the support element distal portion. The fluid sealing plug is compressible. The support element with the fluid sealing plug disposed thereon is configured to be positioned at least partially within the opening through a biologic tissue membrane.

The device may have various optional and/or additional features, including but not limited to features discussed herein. For example, the fluid sealing plug may be generally elongated and disposed along the support element distal portion. The fluid sealing plug may be disposed substantially coaxially circumferentially around the support element distal portion. The fluid sealing plug may be generally in the form of a cylinder, such as an elongated, right circular cylinder. A fluid sealing plug diameter may be substantially constant over a fluid sealing plug length. The support element distal portion may extend distally beyond a distal end portion of the fluid sealing plug and the fluid sealing plug may be absorbent.

In yet another aspect, the invention provides a method of closing an opening through a biologic tissue membrane and sealing against leakage, the method comprising: inserting an implantable fluid sealing plug disposed on an elongated support element into the opening through a biologic tissue membrane, the fluid sealing plug being compressible and absorbent. As possible optional methodology to this method, prior to inserting the fluid sealing plug, a sheath may be positioned to deliver the fluid sealing plug through the sheath and into the opening through the biologic tissue membrane; and/or inserting the fluid sealing plug may comprise inserting the fluid sealing plug and the support element into the sheath. Inserting the fluid sealing plug may comprise inserting a pusher sleeve into the sheath, the support element extending through the pusher sleeve, and the fluid sealing plug may be disposed on the support element distal to the pusher sleeve. After inserting the fluid sealing plug, the method may include withdrawing the sheath proximally off of the fluid sealing plug, the support element, and the pusher sleeve. Inserting the fluid sealing plug may comprise inserting the support element, the fluid sealing plug, and the pusher sleeve into the sheath until a stop disposed on the support element in abutting relation to a pusher sleeve proximal end portion abuts a proximal end of the sheath. The method may further comprise after inserting the fluid sealing plug, removing the stop from the support element; and withdrawing the sheath proximally off of the fluid sealing plug, the support element, and the pusher sleeve.

### BRIEF DESCRIPTION OF THE DRAWINGS

FIG. 1 is an isometric, partial cross-section view of an illustrative fluid sealing plug implanted in a puncture or other opening through a biologic tissue membrane.

FIG. 2 is an elevation view of an illustrative delivery system for a fluid sealing plug.

FIG. 3 is a detailed elevation view of a distal portion of the delivery system with the fluid sealing plug in a dehydrated condition.

FIG. 4 is cutaway view of an illustrative axial stop.

FIG. 5 is an isometric view of an illustrative hydration vial.

FIGS. 6-9 are cutaway views the delivery system and the hydration vial.

FIG. 10 is an isometric view of the delivery system with the fluid sealing plug in a fully hydrated condition.

FIGS. 11-17 are elevation views of an illustrative method using the structural hydrogel fluid sealing plug and associated delivery system.

FIG. 18 is an elevation view of an alternative illustrative delivery system for a fluid sealing plug.

FIG. 19 is a detailed elevation view of a distal portion of the alternative delivery system of FIG. 18 with the fluid sealing plug in a dehydrated condition.

FIGS. 20 and 21 are elevation views of an illustrative method using the structural hydrogel fluid sealing plug and associated delivery system

FIGS. 22-25 are cutaway views of the delivery system and the hydration vial shown in connection with an illustrative method of segmentally hydrating the fluid sealing plug.

FIG. 26 is an isometric view of the fluid sealing plug following segmented hydration.

FIGS. 27-29 are isometric views of alternative example fluid sealing plugs.

### DETAILED DESCRIPTION

Illustrative embodiments according to at least some aspects of the present disclosure are described and illustrated below and include devices and methods relating to medical procedures. Of course, it will be apparent to those of ordinary skill in the art that the embodiments discussed below are examples and may be reconfigured without departing from the scope and spirit of the present disclosure. It is also to be understood that variations of the exemplary embodiments contemplated by one of ordinary skill in the art shall concurrently comprise part of the instant disclosure. However, for clarity and precision, the illustrative embodiments as discussed below may include optional steps, methods, and features that one of ordinary skill should recognize as not being a requisite to fall within the scope of the present disclosure.

The present disclosure includes, among other things, implantable tissue closure devices. Some illustrative embodiments according to at least some aspects of the present disclosure may be used as implantable closure devices for openings, such as punctures, or holes, in biological tissue, such as the meninges membranes. Some illustrative embodiments may reduce and/or prevent leakage of biological fluid, such as cerebrospinal fluid (CSF), through an opening, such as a puncture, e.g., into the soft tissue space (e.g., fat, skin, and/or muscle) that is superficial to the meninges membranes and the CSF system. Generally, some illustrative embodiments may include a fluid sealing plug constructed from a structural hydrogel, which is configured to at least partially obstruct the puncture or opening. While the present detailed description of illustrative embodiments refers to punctures which are generally made during surgical treatment, it will be appreciated that other tissue openings such as natural defects or surgical openings and tissue injuries may be sealed as well. For example, various embodiments according to at least some aspects of the present disclosure may be used to seal a vascular access site, such as may be utilized in an interventional cardiac catherization procedure (e.g., angioplasty, stent delivery), to reduce or prevent undesirable leakage of blood.

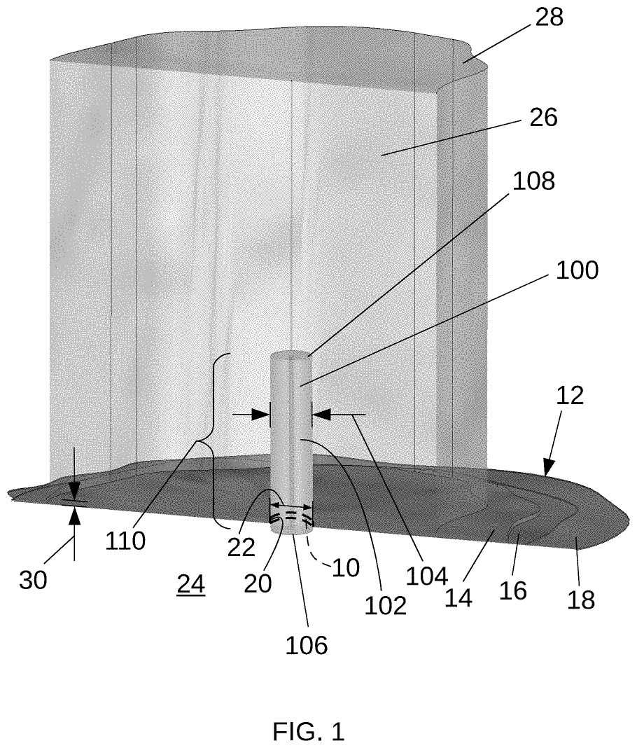

FIG. 1 is an isometric, partial cross-section view of an illustrative fluid sealing plug 100 implanted in a puncture 10 through a biologic tissue membrane, such as the meninges 12, according to at least some aspects of the present disclosure. Generally, the fluid sealing plug 100 may be configured to seal against leakage of a biological fluid (e.g., the CSF) through the sealed opening of the tissue (e.g., the puncture 10). The meninges 12 includes three layers: dura mater 14 (outer/superficial layer), arachnoid mater 16 (middle layer), and pia mater 18 (inner/deep layer). Although some illustrative embodiments are described herein in connection with the meninges and CSF, it is within the scope of the present disclosure to utilize various illustrative closure devices in connection with other biologic tissue membranes and/or to seal openings against leakage of other biological fluids.

As used herein to describe various embodiments from the perspective of a user, “distal” may refer generally to the direction towards the center of a patient's body, and “proximal” may refer generally to the direction away from the center of the patient's body. Depending on the circumstances, “proximal” may also refer to a position closer to the user of the device, while “distal” may refer to a position farther from the user of the device.

Referring to FIG. 1, the fluid sealing plug 100 may be configured to at least partially obstruct or occlude the puncture 10 through the meninges 12. The puncture 10 may be at least partially, such as substantially or fully, sealed by the fluid sealing plug 100 in at least one of the dura mater 14, arachnoid mater 16, and/or pia mater 18. A generally circumferential, radially outer surface 102 of the fluid sealing plug 100, having a fluid sealing plug diameter 104, may engage a generally circumferential, radially inner surface 20 of the puncture 10, having a diameter 22, to provide an at least partially sealed interface between the outer surface 102 of the fluid sealing plug 100 and the inner surface 20 of the puncture 10. As used herein, “diameter” may refer to a major dimension of a shape generally corresponding to the diameter dimension of a circle and is not limited to circular shapes. Also, “diameter” may refer to an exterior dimension, such as the outer diameter of a generally cylindrical object, or to an interior dimension, such as the inner diameter of a tube. Generally, the fluid sealing plug diameter 104 may be selected to generally correspond to diameter 22 of the puncture 10. For example, the fluid sealing plug diameter 104 may approximately match the diameter 22 of the puncture 10. In some alternative embodiments, the fluid sealing plug diameter 104 may be greater than the diameter 22 of the puncture 10, which may facilitate secure engagement of the fluid sealing plug 100 within the puncture 10.

A distal end portion 106 of the fluid sealing plug 100 may protrude into the CSF space 24. That is, the fluid sealing plug 100 may be positioned so that the distal end portion 106 is distal to the biologic tissue membrane (e.g., the meninges 12), such as in the CSF space 24. A fluid sealing plug proximal end portion 108 may be positioned proximal to the meninges 12, such as in the soft tissue 26 (e.g., fat and/or muscle) that overlies the meninges 12 beneath the skin 28. The fluid sealing plug 100 may be generally elongated and has a fluid sealing plug length 110, which is greater than the thickness 30 of the meninges 12. For example, the meninges may be about 0.3 mm thick, on average. In some exemplary embodiments, the fluid sealing plug length 110 may be about 5 mm, such as for closing a lumbar puncture. In other exemplary embodiments, the fluid sealing plug length 110 may be about 10 mm, such as for closing a cervical puncture.

Generally, regardless of its particular form, a fluid sealing plug 100 according to at least some aspects of the present disclosure may reduce and/or prevent fluid flow from one side of the biologic tissue membrane (e.g., meninges 12) to the other side of the biologic tissue membrane through the puncture 10 by sealingly engaging the puncture 10.

The illustrative fluid sealing plug 100 may comprise various types of suitable materials. In some embodiments, the fluid sealing plug 100 may comprise a structural hydrogel. A structural hydrogel, such as may be used to construct the fluid sealing plug 100, differs from a non-structural hydrogel. Generally, a non-structural hydrogel comprises a polymer that can be dehydrated and rehydrated. When rehydrated, the non-structural hydrogel forms a gel, which is easily deformed when an external force is applied to it. A rehydrated hydrogel is generally amorphous, generally lacks the ability to maintain a particular shape without an external supporting structure, and generally assumes the shape of its container. A structural hydrogel also comprises a polymer that can be dehydrated and rehydrated; however, the structural nature of the structural hydrogel distinguishes it from a non-structural hydrogel. Generally, a structural hydrogel is formed in a desired shape during the manufacturing process (e.g., extrusion, molding, casting). When hydrated, the structural hydrogel returns to approximately its originally formed shape and generally retains that shape when external forces are applied. That is, the hydrated structural hydrogel deforms due to the external forces and returns to its original shape. Additionally, a structural hydrogel may be configured to retain its shape, even when immersed in a fluid. For example, the distal end portion 106 of the fluid sealing plug 100 may be in contact with the patient's CSF. Because the fluid sealing plug 100 is constructed from a structural hydrogel, the fluid sealing plug 100 does not disperse within the CSF. In contrast, a non-structural hydrogel would easily disperse within the CSF, which may cause undesired effects. In some illustrative embodiments, the structural hydrogel forming the fluid sealing plug 100 may comprise, for example, a polymer matrix that can readily accept fluid into the matrix and including at least one of amorphous thermoplastic urethane and hydrolyzed polyacrylicnitrile. An exemplary structural hydrogel, when fully hydrated, may comprise about 5% to about 10% solids.

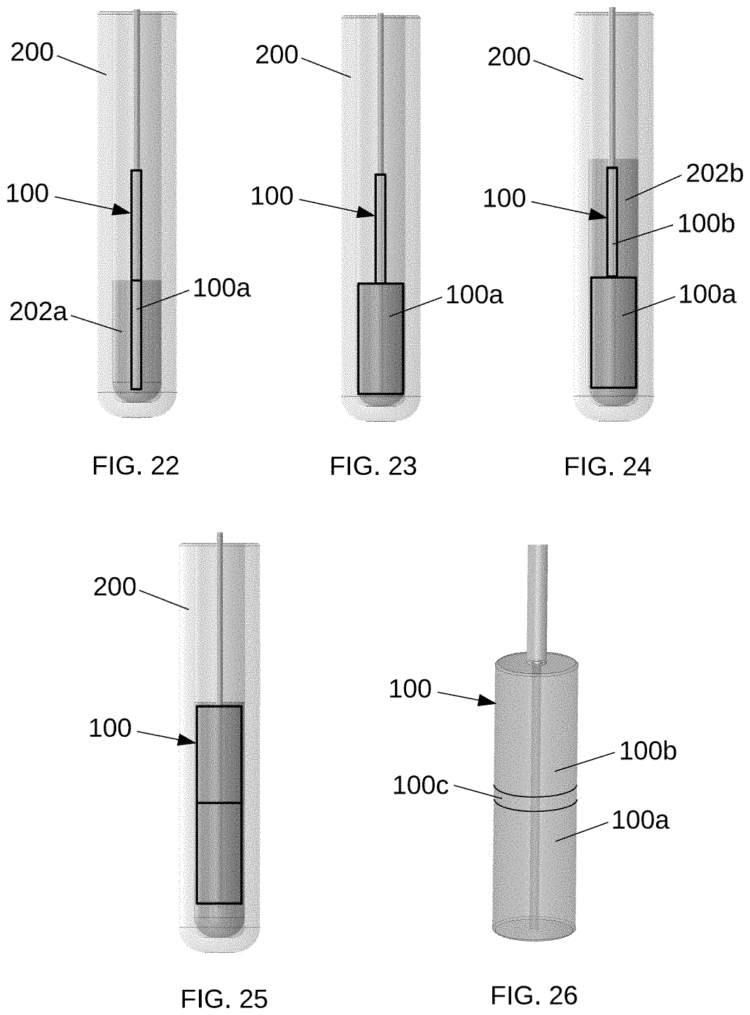

In some exemplary embodiments, the fluid sealing plug 100 may be supplied to the end user with the structural hydrogel in a substantially dehydrated condition. As described below, the fluid sealing plug 100 is configured such that, prior to implantation in the opening 10 through the biologic tissue membrane 12, the structural hydrogel may be at least partially hydrated by the fluid. In some exemplary embodiments, the fluid sealing plug 100 may be supplied to the end user in a partially or fully hydrated condition. In some exemplary embodiments, the fluid sealing plug 100 may be partially hydrated with one fluid and then hydration may be completed using another fluid. For example, the fluid sealing plug 100 may be segmentally hydrated with different fluids as described below with reference to FIGS. 22-25. Or, substantially all of the fluid sealing plug 100 may be partially hydrated with one fluid and then hydration of substantially all of the fluid sealing plug 100 may be completed with another fluid.

In some alternative exemplary embodiments, the illustrative fluid sealing plug 100 may comprise a generally compressible material. In some exemplary embodiments the fluid sealing plug 100 may comprise an absorbent material, which may be capable of absorbing biological and/or non-biological fluids.

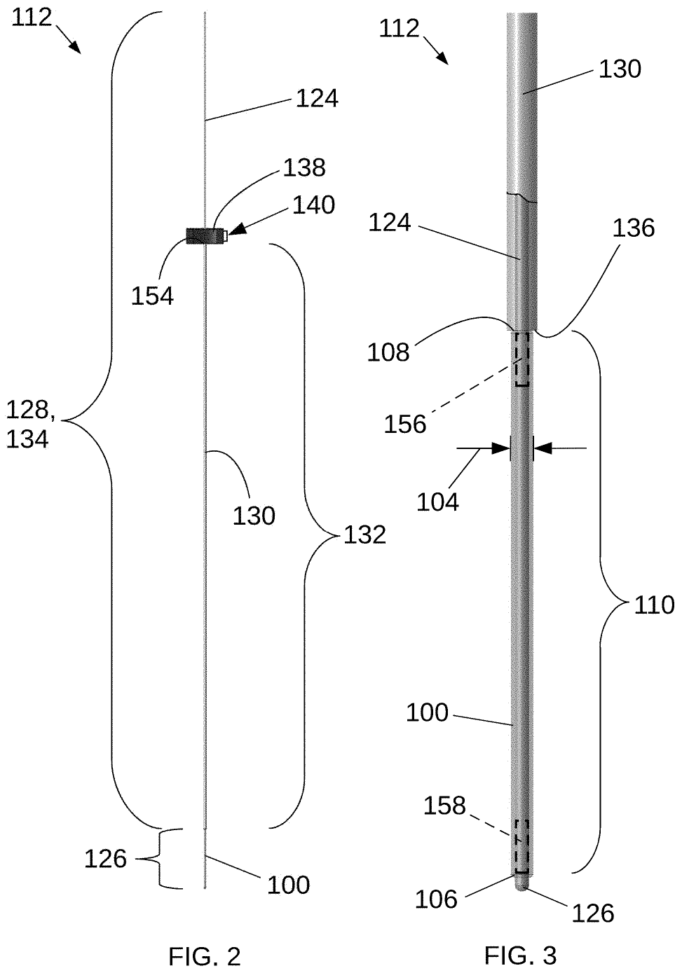

FIG. 2 is an elevation view of an illustrative delivery system 112 for a fluid sealing plug 100 and FIG. 3 is a detailed elevation view of a distal portion of the delivery system 112 with the fluid sealing plug 100 in a dehydrated condition, all according to at least some aspects of the present disclosure. The delivery system 112 comprises an elongated support element 124 including a support element distal portion 126 and a support element proximal portion 128. As described below, the delivery system 112 is configured to facilitate positioning the fluid sealing plug 100 at least partially within the opening 10 through the biologic tissue membrane (e.g., the meninges 12) (FIG. 1).

Referring to FIG. 3, the fluid sealing plug 100 is disposed substantially coaxially circumferentially around and along at least a portion of the support element distal portion 126. In other exemplary embodiments, the fluid sealing plug 100 may be non-coaxially disposed on the support element distal portion 126. For example, the support element distal portion 126 may be laterally offset with respect to the fluid sealing plug 100. In the substantially dehydrated condition shown in FIG. 3, the fluid sealing plug diameter 104 is substantially constant over the fluid sealing plug length 110. In this illustrative embodiment, the support element distal portion 126 extends distally beyond a distal end portion 106 of the fluid sealing plug 100. In other exemplary embodiments, the fluid sealing plug 100 may extend distally beyond the support element distal portion 126.

In the illustrative embodiment of FIGS. 2 and 3, the support element 124 is substantially rigid or semi-rigid. As used herein to describe a support element, “rigid” may refer to a structure that does not significantly deform from its undeformed shape when it is subject to forces that are expected during the intended use of the structure. As used herein to describe a support element, “semi-rigid” may refer to a structure that substantially elastically deforms from its undeformed shape when it is subject to forces that are expected during the intended use of the structure. For example, the support element 124 may comprise a metal wire, such as nickel titanium, stainless steel, tungsten, and/or platinum. Alternatively, the support element may comprise a plastic rod.

Some exemplary embodiments may include one or more markers 156, 158. The markers 156, 158 may be constructed of a material that is substantially detectable, and thus visible to a user, utilizing a medical imaging technique. For example, the markers 156, 158 may be constructed of a radiopaque material for use in connection with fluoroscopic imaging techniques and/or an echogenic material for use in connection with ultrasound imaging techniques. In some example embodiments, a marker 156, 158 may be configured so that both its position and orientation may be determined via the medical imaging technique, such as by including non-symmetric and/or non-uniform geometric features. Accordingly, the position and/or orientation of the delivery system 112 may be ascertained using the imaging technique. In some example embodiments, the markers 156, 158 may be positioned so that they indicate the fluid sealing plug length 110, which may facilitate proper placement of the plug 100 using fluoroscopy. Some embodiments, such as those comprising a generally radiopaque support element 124, may not include one or both markers 156, 158.

The illustrative delivery system 112 further comprises an elongated, tubular pusher sleeve 130 slidably disposed coaxially circumferentially around and along at least a portion of the support element proximal portion 128. In other exemplary embodiments, such as those including fluid sealing plugs 100 that are not coaxially disposed on the support element 124, the pusher sleeve 130 may not be coaxially disposed on the support element proximal portion 128. In some such embodiments, the pusher sleeve 130 may be laterally offset corresponding to the lateral offset of the fluid sealing plug 100. The pusher sleeve 130 has a pusher sleeve length 132, which may be less than a support element proximal portion length 134. A pusher sleeve distal end portion 136 is disposed in abutting relation to the fluid sealing plug proximal end portion 108. In some exemplary embodiments, the pusher sleeve 130 may be constructed from various polymeric materials (e.g., polyether block amide, urethane, nylon), which may have durometers above about 55D. In other embodiments, the pusher sleeve 130 may be constructed from metal, such as stainless steel or nickel titanium.

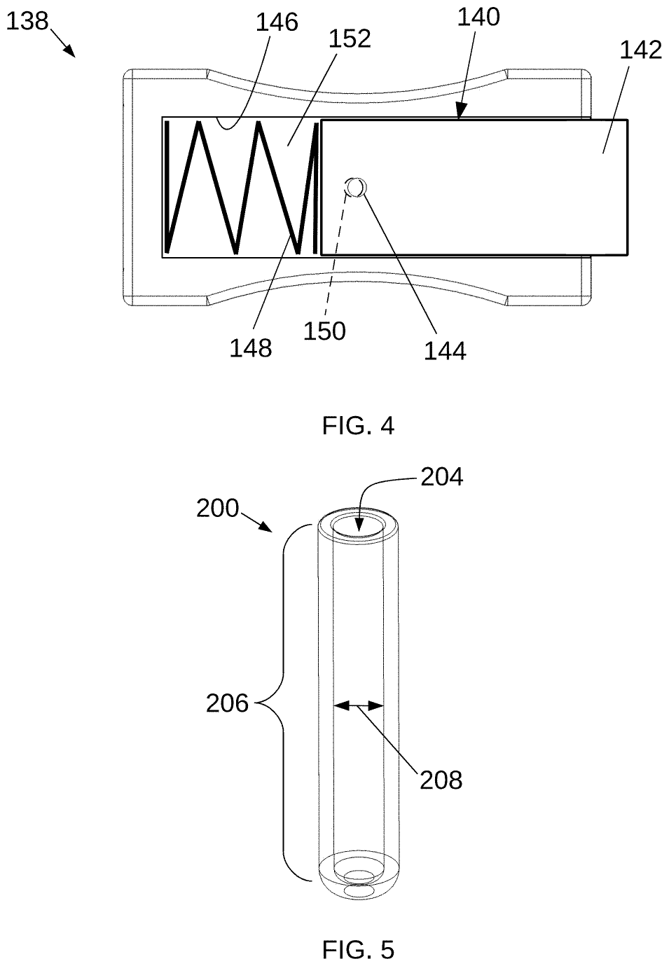

FIG. 4 is cutaway view of an illustrative axial stop 138, according to at least some aspects of the present disclosure. Referring to FIGS. 2 and 4, the illustrative delivery system 112 further comprises an axial stop 138 releasably engaged with the support element proximal portion 128. The stop 138 is selectively axially slidable on the support element proximal portion 128. The stop 138 is configured to selectively oppose axial movement (e.g., proximal movement) of the pusher sleeve 130 relative to the support element 124. Specifically, the stop 138 is configured to selectively oppose proximal movement of the pusher sleeve 130 relative to the support element 124 by selectively abutting a pusher sleeve proximal end portion 154. Accordingly, the stop 138 is configured to selectively hold the pusher sleeve distal end portion 136 in abutting relation with the proximal end portion of the fluid sealing plug 108.

The stop 138 comprises an engagement mechanism 140 configured to selectively releasably engage the support element proximal portion 128. The engagement mechanism 140 comprises a slide 142, which includes a through hole 144 configured to receive the support element 124 therethrough. The slide 142 is laterally slidably disposed in a cavity 146 within the stop 138. A spring 148 (e.g., a compression spring) is disposed within the cavity 146 and biases the slide 142 such that the support element 124 is selectively secured by the spring-biased offset of the through hole 144 of the slide and a corresponding through hole 150 through a wall 152 of the stop 138. Pressing the slide 142 inward (e.g., to compress the spring 148) aligns the hole 144 of the slide 142 with the hole 150 of the wall 152, thereby allowing axial movement of the stop 138 along the support element 124. Releasing the slide 142, thereby allowing the spring 148 to push the slide 142 outward, secures the support element 124 between the offset holes 144, 150.

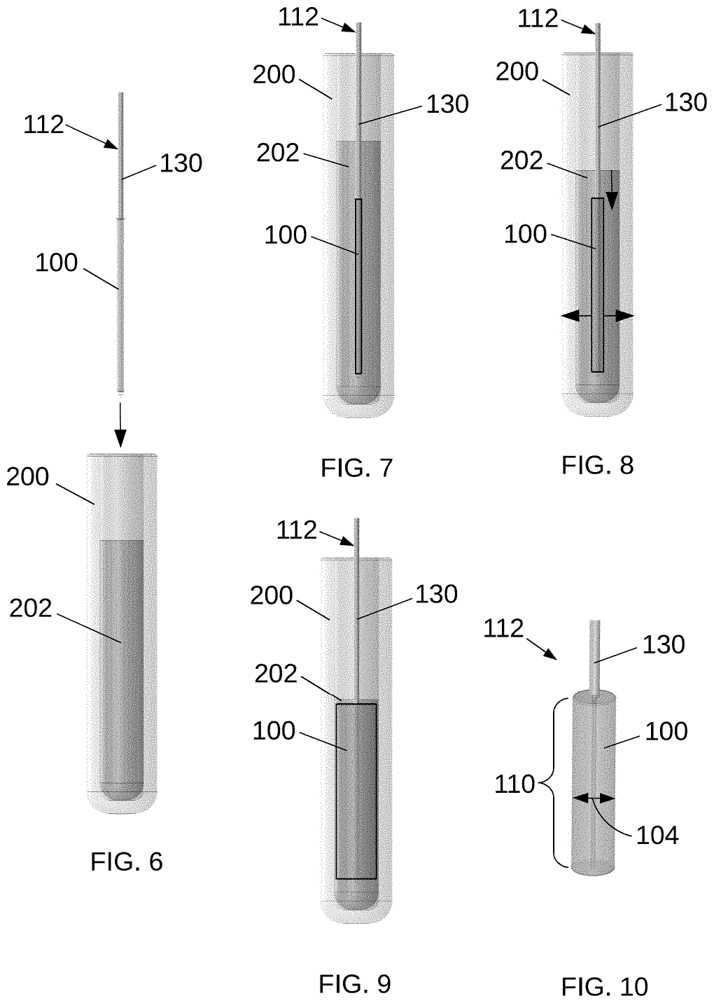

FIG. 5 is an isometric view of an illustrative hydration vial 200 and FIGS. 6-9 are cutaway views the delivery system 112 and the hydration vial 200, all according to at least some aspects of the present disclosure. Referring to FIGS. 2, 3, and 5-9, the hydration vial 200 is configured to receive a fluid 202 and the support element distal portion 126 and the fluid sealing plug 100 therein. In this illustrative embodiment, the hydration vial 200 comprises an interior cavity 204 that is generally in the form of a right circular cylinder. The interior cavity 204 has an interior length 206 that is longer than the fluid sealing plug length 110. In this illustrative embodiment, the interior cavity 204 has an interior diameter 208 that is greater than the fluid sealing plug diameter 104 when the fluid sealing plug 100 is fully hydrated. In other exemplary embodiments, the hydration vial 200 may have other shapes, such as generally bowl-like. It will be appreciated, however, that fluid remaining in the hydration vial 200 after the desired hydration of the fluid sealing plug 100 has been achieved may be wasted. Thus, hydration vials 200 having interior cavities 204 that generally correspond to the size and shape of the fluid sealing plug 100 may reduce the amount of fluid 200 that is necessary to hydrate the fluid sealing plug 100.

FIGS. 6-9 illustrate an exemplary hydration process. Referring to FIG. 6, the fluid 202 is placed in the hydration vial 200 in preparation for receiving the delivery system 112 comprising the dehydrated fluid sealing plug 100. Referring to FIG. 7, the dehydrated fluid sealing plug 100 is at least partially immersed in the fluid 202 in the hydration vial 200. Referring to FIG. 8, the structural hydrogel of the fluid sealing plug 100 is partially hydrated. The level of the fluid 202 in the hydration vial 200 drops and the diameter of the fluid sealing plug 100 increases as the fluid 202 is absorbed into the fluid sealing plug 100. Referring to FIG. 9, the structural hydrogel of the fluid sealing plug 100 is substantially fully hydrated.

FIG. 10 is an isometric view of the delivery system 112 with the fluid sealing plug 100 in a fully hydrated condition, according to at least some aspects of the present disclosure. In the illustrative embodiment, the structural hydrogel of the fluid sealing plug 100 is configured such that, when fully hydrated, the fluid sealing plug 100 is generally in the form of an elongated, right circular cylinder. In this embodiment, the fluid sealing plug diameter 104 is substantially constant over the fluid sealing plug length 110 when the structural hydrogel comprising the fluid sealing plug is fully hydrated.

Referring to FIGS. 6-10, the amount of fluid 202 placed in the hydration vial 100 may be selected to provide the desired hydration results, and the fluid 202 may be measured before it is placed into the vial. For example, in some circumstances, it may be desirable to implant the fluid sealing plug 100 in a partially hydrated condition. In such a partially hydrated condition, the fluid sealing plug diameter 104 may be between that of the diameter when the fluid sealing plug 100 is dehydrated (e.g., FIG. 6) and the diameter when the fluid sealing plug 100 is fully hydrated (e.g., FIG. 10). Also, in some partially hydrated conditions, the stiffness of the fluid sealing plug 100 may be between that of the fully dehydrated condition and the fully hydrated condition. Specifically, in a partially hydrated condition, the fluid sealing plug 100 may have a smaller diameter and may be more rigid than when it is in the fully hydrated condition. In some exemplary embodiments, a delivery system 112 may be supplied with a hydration table listing specific volumes of fluid 202 that should be placed in the hydration vial 100 to achieve desired various fluid sealing plug diameters 104. In some circumstances, implanting a partially hydrated fluid sealing plug 100 may allow the fluid sealing plug 100 to continue to expand to its fully hydrated state after implantation, such as due to absorption of fluid present at the implantation site.

Generally, the fluid 202 may comprise any biocompatible liquid that is at least partially absorbable into the structural hydrogel of the fluid sealing plug 100. For example, the fluid 202 may include one or more of water, saline solution, blood, amniotic fluid, cerebrospinal fluid, and polyethylene glycol. In some exemplary embodiments, the fluid 202 may include a thrombolytic agent, such as anistreplase, streptokinase, kabikinase, or reteplase. In some exemplary embodiments, the fluid 202 may comprise a contrast agent, such as iodine, or barium sulfate, or small particles of tungsten or barium (e.g., suspended in the fluid). to enhance the radiopacity of the fluid sealing plug 100. In some exemplary embodiments, it may be desirable to hydrate the fluid sealing plug 100 using only fluids that will be absorbed into the tissue surrounding the implantation site so that, eventually, only the structural hydrogel polymer matrix remains at the implantation site. This may facilitate future reintervention using the same access site.

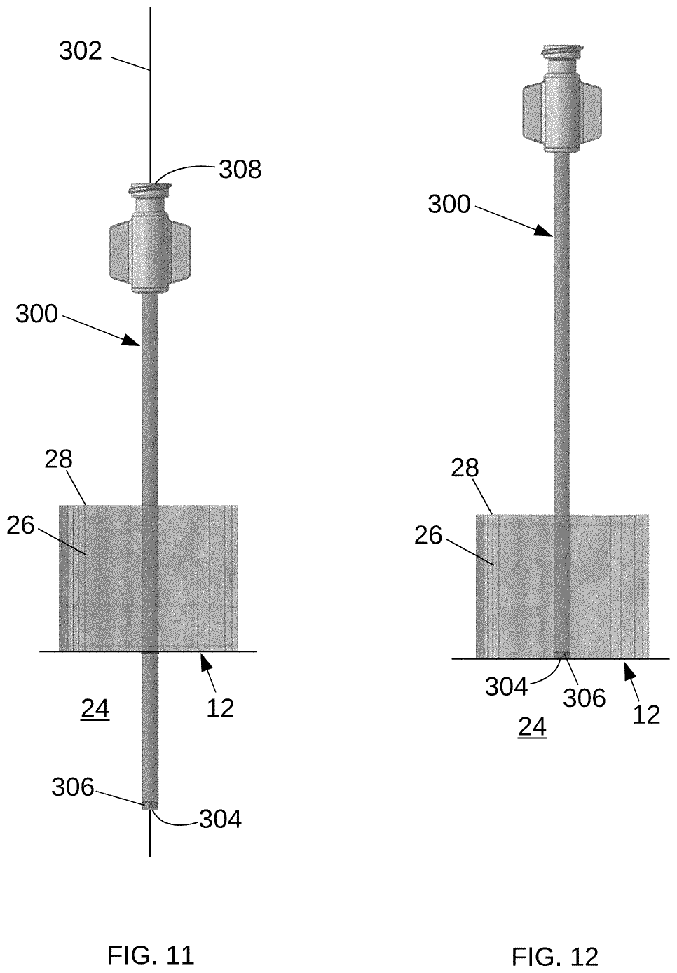

Exemplary methods of using a fluid sealing plug 100 according to at least some aspects of the present disclosure are described below with reference to FIGS. 11-17, which may include optional and/or alternative structures and/or operations. FIGS. 11-17 are elevation views of an illustrative method using the structural hydrogel fluid sealing plug 100 and associated delivery system 112, all according to at least some aspects of the present disclosure. Although FIGS. 11-17 and the corresponding description focus on the use of the fluid sealing plug 100 to seal the puncture 10 through the meninges 12 to prevent and/or reduce leakage of CSF, it will be appreciated that generally similar operations may be utilized when alternative embodiment closure devices are used to seal openings though other biologic tissues to prevent and/or reduce leakage of other biological fluids (e.g., blood).

Referring to FIG. 11, a generally tubular sheath 300 is positioned to provide access to an internal biological fluid system (e.g., the CSF space 24) from an exterior of a patient. The sheath extends from external to the patient's skin 28, through the soft tissue 26, and through a biologic tissue membrane (e.g., the meninges 12). The sheath 300 or other devices may be inserted over a guidewire 302, which may extend through a central, longitudinal lumen of the sheath 300. The guidewire 302 may be placed using a hollow needle (not shown) using standard techniques. The longitudinal lumen of the sheath may serve as an introducer for other devices, such as catheters, which may be utilized during an interventional procedure, such as to treat defects that may be present within the CSF system. Further, the sheath 300 is configured to deliver the fluid sealing plug 100 to the opening 10 (FIG. 1), as described below. The sheath 300 includes a distal end 304, including a distal end opening. In some exemplary embodiments the sheath may include a radiopaque marker 306, such as proximate the distal end 304. The sheath 300 includes a proximal end 308, including a proximal end opening. In some exemplary procedures, the user may remove a small amount of cerebrospinal fluid for laboratory analysis and/or the user may inject a pharmaceutical into the CSF space 24.

Once the need for access to the CSF space 24 via the puncture 10 is finished, implantation of the fluid sealing plug 100 may begin. Referring to FIG. 12, the guidewire 302 is removed (it not already removed) and the sheath 300 is repositioned proximally so that the distal end 304 is aligned with the meninges 12. This may be facilitated by using a medical imaging technique, such as fluoroscopy, to view the position of the marker 306.

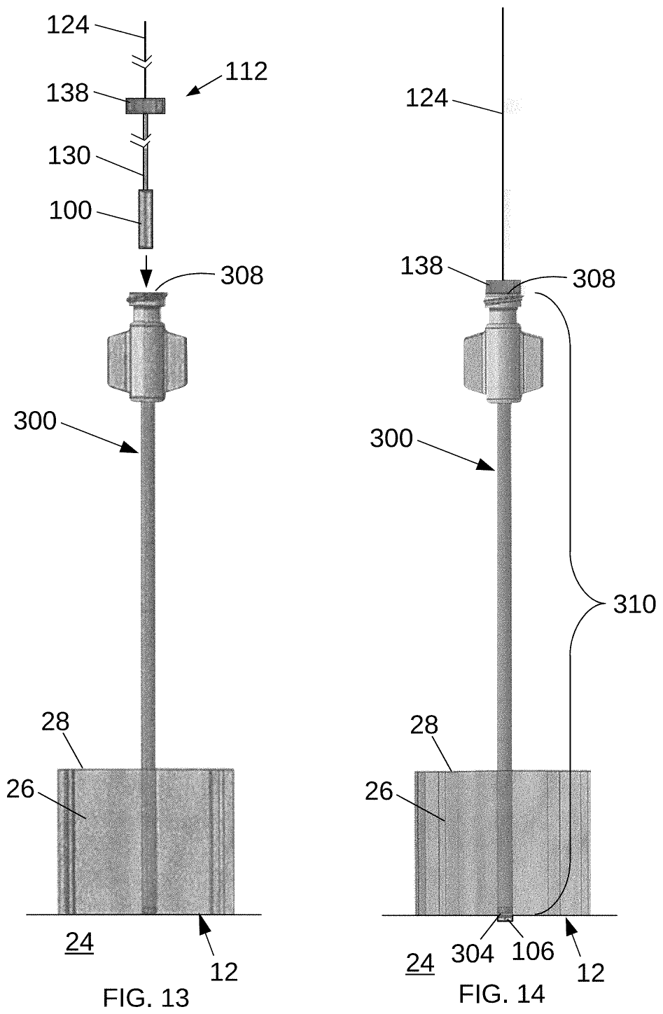

Referring to FIGS. 13 and 14, the delivery system 112, including the fluid sealing plug 100, is inserted into the sheath 300 via the proximal end 308. Prior to insertion into the sheath 300, the fluid sealing plug 100 may be partially or fully hydrated, such as described above with reference to FIGS. 6-10. The fluid sealing plug diameter 104 (FIG. 10) may be selected based upon the lumen size of the sheath 30. For example, a fluid sealing plug 100 having the desired fluid sealing plug diameter 104 when fully hydrated may be used. Alternatively, a fluid sealing plug 100, such as a fluid sealing plug 100 having a larger fluid sealing plug diameter 104 when fully hydrated, may be partially hydrated to achieve the desired fluid sealing plug diameter 104. For example, when using a sheath 300 having a relatively thick wall, it may be desirable to insert the fluid sealing plug 100 in a partially hydrated condition so that it may be easily advanced through the lumen of the sheath. Then, after it is implanted, the fluid sealing plug 100 may continue to hydrate with the patient's biological fluid until it reaches a fully hydrated condition.

When fully inserted, the stop 138 abuts the proximal end 308 of the sheath 300 and the distal end portion 106 of the fluid sealing plug 100 extends beyond the distal end 304 of the sheath 300 and through the meninges 12. More specifically, referring to FIGS. 2 and 14, the pusher sleeve length 132, the fluid sealing plug length 110, and/or the sheath length 310 are configured so that the distal end portion 106 of the fluid sealing plug 100 extends beyond the distal end 304 of the sheath 300 when the delivery system 112, including the fluid sealing plug 100, the support element 124, and the pusher sleeve 130, is fully inserted into the sheath 300. When fully inserted, the fluid sealing plug 100 extends through the meninges 12 so that the distal end portion 106 is in the CSF space 24, and the entire pusher sleeve length 132 is within the sheath 300.

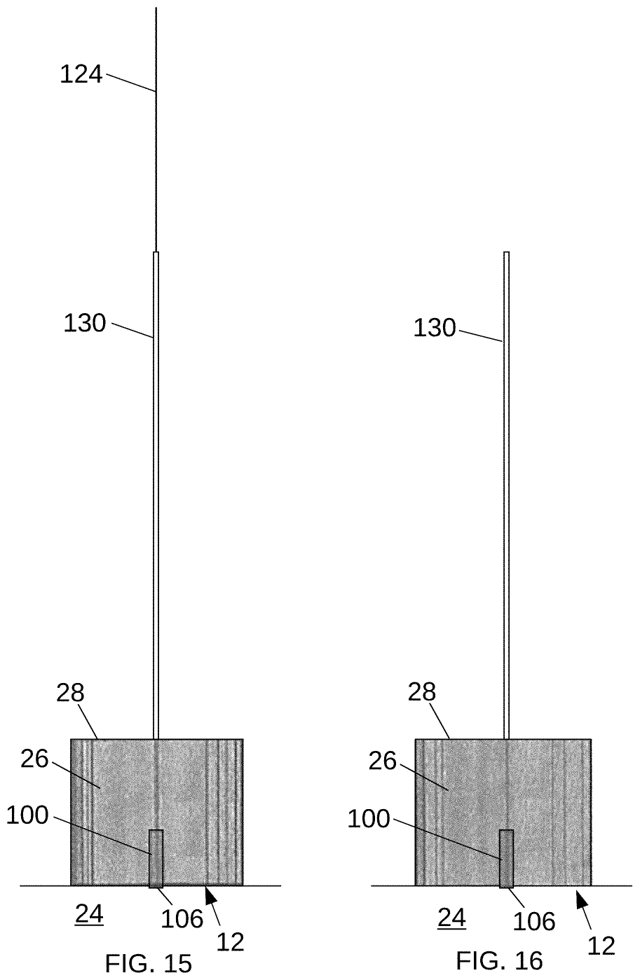

Referring to FIG. 15, the stop 138 and sheath 300 are withdrawn proximally, leaving the fluid sealing plug 100, the support element 124, and the pusher tube 130 in place. The fluid sealing plug 100 remains extending through the meninges 12, with the distal end portion 106 in the CSF space 24. The fluid sealing plug proximal end portion 108 is positioned within the soft tissue 26 beneath the skin 28. In some procedures, the position of the fluid sealing plug 100 may be verified and/or the fluid sealing plug 100 may be monitored to determine its efficacy of closing the puncture 10. If the fluid sealing plug 100 is not positioned as desired and/or does not satisfactorily seal the puncture 10, it may be repositioned or removed.

Referring to FIG. 16, once the user is satisfied with the implantation of the fluid sealing plug 100, the support element 124 is detached from the fluid sealing plug 100, and the support element 124 is withdrawn proximally through the pusher sleeve 130, leaving the fluid sealing plug 100 and the pusher tube 130 in place. Generally, the fluid sealing plug 100 and the support element 124 are configured so that the frictional force between them is high enough to prevent unintended separation but is low enough to allow separation using the pusher sleeve 130 when desired. In some exemplary embodiments, the support element 124 may include one or more engaging elements, which may be configured to increase the frictional force between the fluid sealing plug 100 and the support element 124.

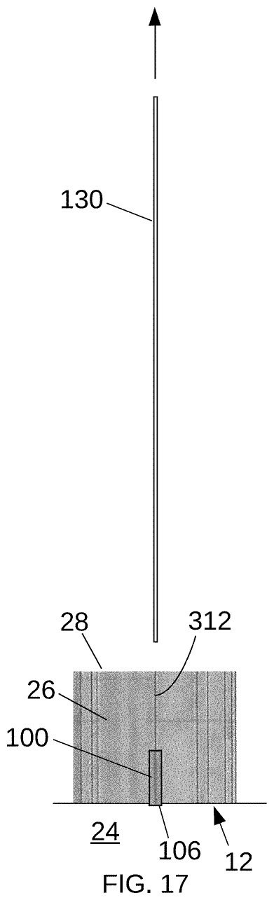

Referring to FIG. 17, the pusher tube 130 is withdrawn proximally, leaving the fluid sealing plug 100 in place. After removing the pusher tube 130, an access channel 312 (e.g., wound) may remain the in soft tissue 26 and the skin 28. The wound remaining in the soft tissue 26 and the skin 28 may seal spontaneously, such as due to the elastic properties of the soft tissue 26 and/or the skin 28. In some procedures, the wound may be closed (e.g., sutured) and/or bandaged. Referring to FIGS. 1 and 17, the fluid sealing plug 100 may reduce and/or prevent flow of CSF from the CSF space 24 into the access channel 312 through the meninges 12 via the puncture 10 by sealingly engaging the puncture 10. The fluid sealing plug 100 provides radial compression against the soft tissue 26 and meninges 12 equivalent to the radially inward forces exerted on the fluid sealing plug 100 by those structures.

Various steps of the implantation process of the fluid sealing plug 100 described above may be conducted using clinically acceptable visualization techniques (e.g. fluoroscopy, endoscopy, a computed tomography scan, magnetic resonance imaging, ultrasound, etc.) as desired by the user. Generally similar methods and/or structures may be used to deliver and/or deploy alternative embodiment closure devices according to at least some aspects of the present disclosure.

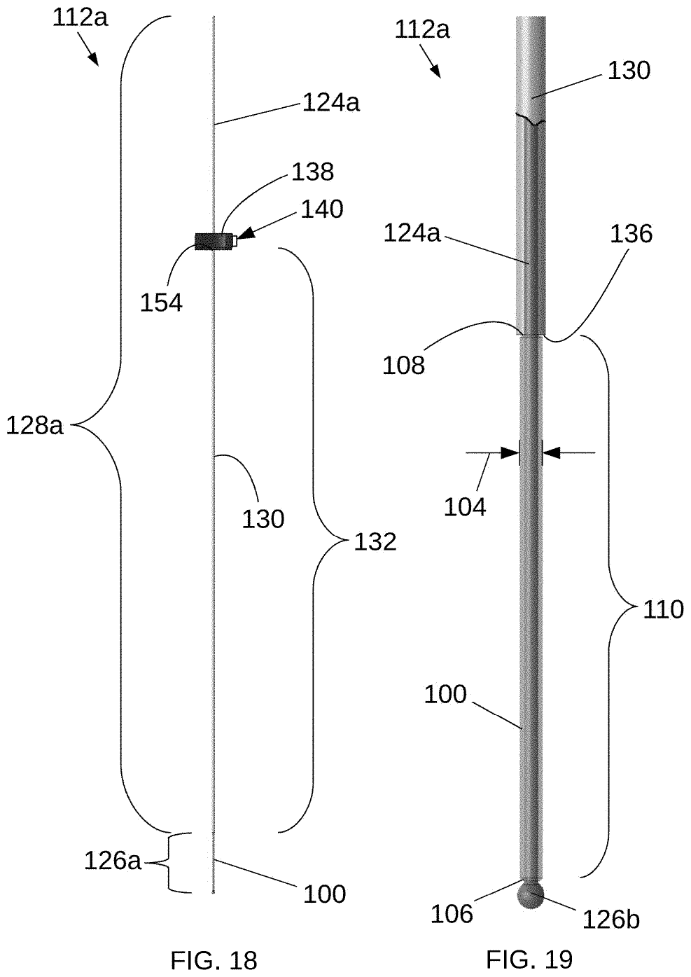

FIG. 18 is an elevation view of an alternative illustrative delivery system 112a for a fluid sealing plug 100 and FIG. 19 is a detailed elevation view of a distal portion of the alternative delivery system 112a with the fluid sealing plug 100 in a dehydrated condition, all according to at least some aspects of the present disclosure. The delivery system 112a shown and described below with respect to FIGS. 18-21 is generally similar to the delivery system 112 shown and described above with respect to FIGS. 1-17 and is configured to facilitate positioning the fluid sealing plug 100 at least partially within the opening 10 through the biologic tissue membrane (e.g., the meninges 12) (FIG. 1). Generally, the delivery system 112a described below differs from the delivery system 112 described above primarily in that a generally flexible support element 124a has been substituted for the generally rigid or semi-rigid support element 124. In the description below, like reference numerals refer to like structure shown and described above. Unless specifically indicated, the description of the structure and function or methodology of corresponding components with respect to the delivery system 112 generally applies to the delivery system 112a. Therefore, repeated explanation of previously described structure and function or methodology is not necessary.

Referring to FIGS. 18 and 19, the delivery system 112a comprises an elongated support element 124a including a support element distal portion 126a and a support element proximal portion 128a. In this illustrative embodiment, the support element 124a is substantially flexible. As used herein to describe a support element, “flexible” may refer to a structure that is substantially non-rigid and/or does not significantly resist bending and/or buckling when it is subject to forces that are expected during use of the structure as intended. For example, the support element 124a may comprise one or more filaments, such as suture material. In some exemplary embodiments, the filaments (e.g., suture material) may be bioabsorbable. The support element 124a may be constructed from, for example, polyglycolic acid, polylactic acid, polydioxanone, or caprolactone.

In this illustrative embodiment, the fluid sealing plug 100 is disposed substantially coaxially circumferentially around and along at least a portion of the support element distal portion 126a. In some alternative exemplary embodiments, the fluid sealing plug 100 may be disposed non-coaxially on the support element distal portion 126a, as discussed above. In the substantially dehydrated condition, the fluid sealing plug diameter 104 is substantially constant over the fluid sealing plug length 110. In this illustrative embodiment, the support element distal portion 126a extends distally beyond a distal end portion 106 of the fluid sealing plug 100 and comprises a retainer 126b. In some alternative exemplary embodiments, the distal end portion 106 of the fluid sealing plug 100 may extend distally beyond the support element distal portion 126a and/or the retainer 126b, as discussed above. In this illustrative embodiment, in which the support element 124a comprises a flexible, bioabsorbable suture, the retainer 126b comprises a knot (e.g., a stopper knot). In alternative exemplary embodiments, the retainer 126b may comprise, for example, a polymer sphere or disk (or other shape). Some exemplary embodiments may include one or more markers similar to markers 156, 158 described above.

In this illustrative embodiment including a flexible support element 124a, the fluid sealing plug 100 is compressed longitudinally between the pusher sleeve distal end portion 136 (pushing distally) and the retainer 126b (pushing proximally). In combination with the structured nature of the structural hydrogel forming the fluid sealing plug 100, this allows the fluid sealing plug 100 to assume and maintain a generally longitudinally straight arrangement with respect to the pusher sleeve 130 for delivery into the patient.

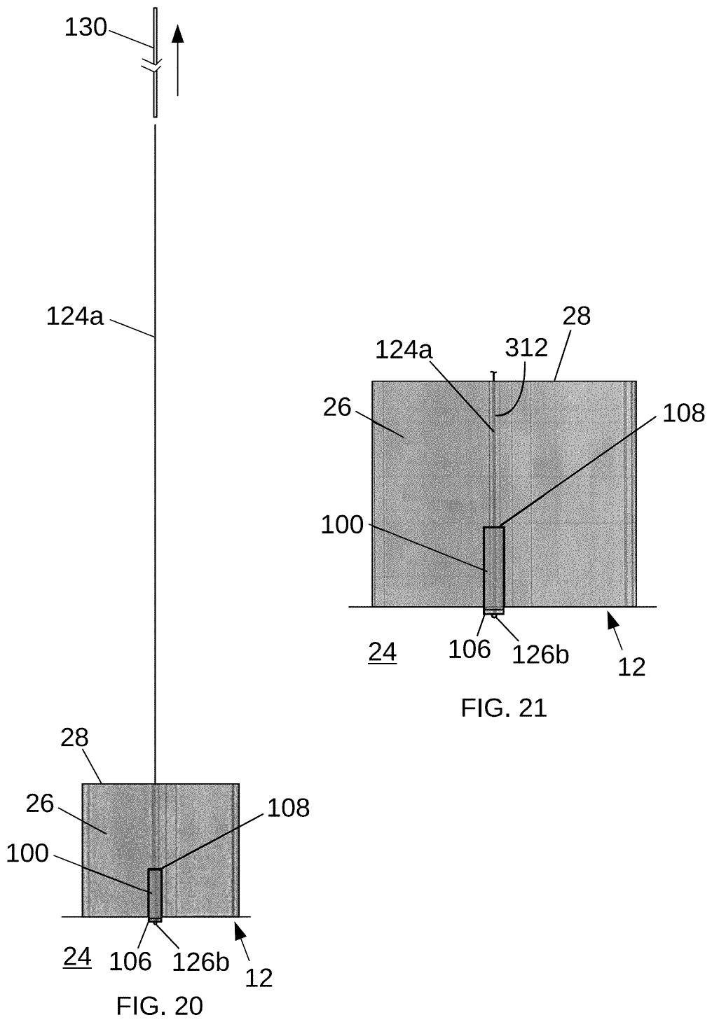

Exemplary methods of using a delivery system 112a according to at least some aspects of the present disclosure are described below with reference to FIGS. 11-15 and 18-21, which may include optional and/or alternative structures and/or operations. FIGS. 20 and 21 are elevation views of an illustrative method using the structural hydrogel fluid sealing plug 100 and associated delivery system 112a, all according to at least some aspects of the present disclosure. Although the following description and corresponding figures focus on the use of the fluid sealing plug 100 to seal the puncture 10 through the meninges 12 to prevent and/or reduce leakage of CSF, it will be appreciated that generally similar operations may be utilized when alternative embodiment closure devices are used to seal openings though other biologic tissues to prevent and/or reduce leakage of other biological fluids (e.g., blood).

Exemplary methods of using the delivery system 112a may include the operations substantially similar to those described above with reference to the delivery system 112 and FIGS. 11-15. Following those operations, and referring to FIG. 20, the pusher sleeve 130 is withdrawn proximally off of the support element 124a, leaving the support element 124a and the fluid sealing plug 100 in place. The fluid sealing plug 100 remains extending through the meninges 12, with the distal end portion 106 in the CSF space 24. The fluid sealing plug proximal end portion 108 is positioned within the soft tissue 26 beneath the skin 28.

Referring to FIG. 21, the support element 124a is cut near (e.g., at or slightly below) the surface of the skin 28 while the support element 124a remains attached to the fluid sealing plug 100. The remaining portion of the support element 124a and the attached fluid sealing plug 100 remain implanted in the patient. The support element 124a extends within the access channel 312 (e.g., wound) in the in soft tissue 26 and the skin 28. The wound remaining in the soft tissue 26 and the skin 28 may seal spontaneously, such as due to the elastic properties of the soft tissue 26 and/or the skin 28. In some procedures, the wound may be closed (e.g., sutured) and/or bandaged. The fluid sealing plug 100 may reduce and/or prevent flow of CSF from the CSF space 24 into the access channel 312 through the meninges 12 via the puncture 12 by sealingly engaging the puncture 10. In embodiments including a bioabsorbable support element 124a, the support element 124a may be absorbed by the patient's tissue over time, leaving only the fluid sealing plug 100.

FIGS. 22-25 are cutaway views of the delivery system 112 and the hydration vial 200 shown in connection with an illustrative method of segmentally hydrating the fluid sealing plug 100 and FIG. 26 is an isometric view of the fluid sealing plug 100 following segmented hydration, all according to at least some aspects of the present disclosure. Various operations in this illustrative method are generally similar to those described above with reference to FIGS. 6-10. Generally, the methods described below differ from the operations described above primarily in that one or more portions of the fluid sealing plug 100 are at least partially hydrated with a fluid that differs from a fluid that is used to hydrate another portion of the fluid sealing plug 100. In the description below, like reference numerals refer to like structure shown and described above. Unless specifically indicated, the description of the structure and function or methodology of corresponding components generally applies. Therefore, repeated explanation of previously described structure and function or methodology is not necessary.

Referring to FIG. 22, a first fluid 202a is placed in the hydration vial 200, and a first portion 100a of the fluid sealing plug 100 is immersed in the first fluid 202a. Referring to FIG. 23, the first portion 100a of the fluid sealing plug 100 partially or fully hydrates by absorbing the first fluid 202a. Referring to FIG. 24, a second fluid 202b is placed in the hydration vial 200, and a second portion 100b of the fluid sealing plug 100 is immersed in the second fluid 202b. Referring to FIG. 25, the second portion 100b of the fluid sealing plug 100 partially or fully hydrates by absorbing the second fluid 202b. Referring to FIG. 26, the resulting fluid sealing plug 100 comprises the first portion 100a hydrated with the first fluid 202a and the second portion 100b hydrated with the second fluid 202b. Some embodiments may include a transition portion 100c between the first portion 100a and the second portion 100b into which substantial amounts of both the first fluid 202a and the second fluid 202b have been absorbed. In this exemplary process, both the first portion 100a and the second portion 100b are immersed in the second fluid 202b as shown in FIG. 24. But, because the first portion 100a was substantially fully hydrated by the first fluid 202a, the first portion 100a may absorb little, if any, of the second fluid 202b. In an exemplary embodiment, the first fluid 202a may comprise CSF and the second fluid 202b may comprise blood. In another exemplary embodiment, the first fluid 202a may comprise a contrast agent and the second fluid 202b may not comprise a contrast agent. Such a selection of fluids 202a, 202b may be utilized, for example, in an embodiment in which the fluid sealing plug 100 extends distally beyond the support member 124 because the first fluid 202a comprising the contrast agent may allow visualization of the distal end portion 106 of the fluid sealing plug.

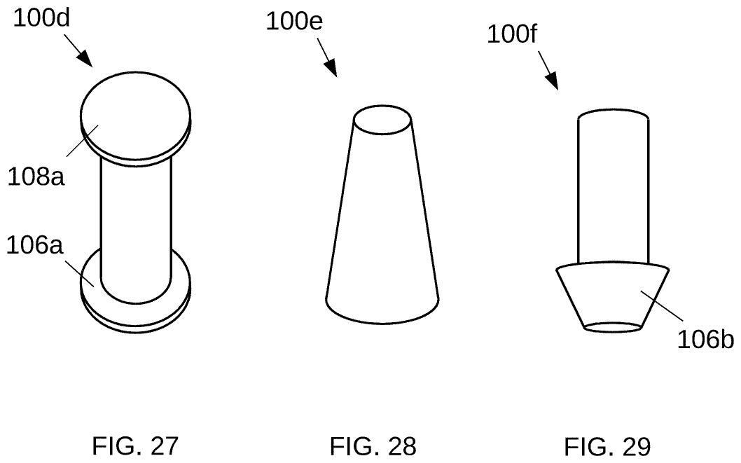

FIGS. 27-29 are isometric views of alternative example fluid sealing plugs 100d, 100e, 100f, all according to at least some aspects of the present disclosure. FIG. 27 shows a fluid sealing plug 100d, which may include one or more generally radially extending flanges, such as a distal flange 106a and/or a proximal flange 108a. FIG. 28 shows a fluid sealing plug 100e, which is formed in a generally tapered shape, such as generally the shape of a truncated cone. It will be appreciated that tapered fluid sealing plugs may be oriented with the wider portion proximally or distally. FIG. 29 shows a fluid sealing plug 100f with a generally barb-like, distal engaging element 106b, which may be configured to engage a biologic tissue in which the fluid sealing plug 100f is implanted. Various features of fluid sealing plugs 100d, 100e, 100f may be utilized in other embodiments according to at least some aspects of the present disclosure.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A device for sealing an opening through a biologic tissue membrane against leakage, the device comprising:
an implantable fluid sealing plug including a structural hydrogel;
wherein the fluid sealing plug is configured to be positioned at least partially within the opening through a biologic tissue membrane,
the fluid sealing plug is configured to increase in diameter upon absorbing a fluid, and
the increase in diameter of the fluid sealing plug upon absorbing the fluid seals the opening through a biologic tissue membrane.

2. The device of claim 1, wherein the fluid sealing plug is configured such that, when in a substantially fully hydrated condition, the fluid sealing plug is generally in the form of an elongated, right circular cylinder.

3. The device of claim 1, further comprising an elongated support element including a support element distal portion and a support element proximal portion;
wherein the fluid sealing plug is disposed on the support element distal portion, and
the support element with the fluid sealing plug disposed thereon is configured to be positioned at least partially within the opening through the biologic tissue membrane.

4. The device of claim 3, wherein the fluid sealing plug is disposed substantially coaxially circumferentially around and along the support element distal portion.

5. The device of claim 3, wherein the support element distal portion extends distally beyond a distal end portion of the fluid sealing plug.

6. The device of claim 3, further comprising an elongated, tubular pusher sleeve slidably disposed circumferentially around and along at least a portion of the support element proximal portion.

7. The device of claim 6, wherein a pusher sleeve distal end portion is disposed in abutting relation to a fluid sealing plug proximal end portion.

8. The device of claim 6, further comprising an axial stop releasably engageable with the support element proximal portion to selectively oppose proximal movement of the pusher sleeve relative to the support element.

9. The device of claim 8, wherein the axial stop is configured to selectively oppose proximal movement of the pusher sleeve relative to the support element by selectively abutting a pusher sleeve proximal end portion.

10. The device of claim 8, wherein the axial stop is configured to selectively hold a pusher sleeve distal end portion in abutting relation with a proximal end portion of the fluid sealing plug.

11. The device of claim 8, wherein the axial stop is selectively axially slidable on the support element proximal portion.

12. The device of claim 8, wherein the axial stop comprises an engagement mechanism configured to selectively releasably engage the support element proximal portion.

13. The device of claim 12, wherein the engagement mechanism comprises a spring and a slide.

14. The device of claim 3, further comprising a hydration vial configured to receive therein the fluid and the support element distal portion with the fluid sealing plug disposed thereon.

15. The device of claim 3, further comprising a sheath configured to deliver the fluid sealing plug to the opening through the biologic tissue membrane;
wherein the sheath is configured to receive the fluid sealing plug therethrough; and
the sheath is configured to receive at least a portion of the support element therein.

16. A device for sealing an opening through a biologic tissue membrane against leakage, the device comprising:
an elongated support element comprising a support element distal portion and a support element proximal portion; and
an implantable fluid sealing plug disposed on the support element distal portion, the fluid sealing plug being compressible;
wherein the support element with the fluid sealing plug disposed thereon is configured to be positioned at least partially within the opening through a biologic tissue membrane, the sealing plug sealingly engaging the opening.

17. The device of claim 16, wherein the fluid sealing plug is generally in the form of an elongated, right circular cylinder.

18. The device of claim 16, wherein the support element distal portion extends distally beyond a distal end portion of the fluid sealing plug.

19. A method of closing an opening through a biologic tissue membrane and sealing against leakage, the method comprising:
implanting a fluid sealing plug in the opening through a biologic tissue membrane;
wherein the fluid sealing plug comprises a structural hydrogel,
a diameter of the fluid sealing plug increases upon absorbing a fluid, and
the increase in diameter of the fluid sealing plug upon absorbing the fluid seals the opening through a biologic tissue membrane.

20. The method of claim 19, wherein the fluid sealing plug is disposed on an elongated support element, and
the method further comprising:
prior to inserting the fluid sealing plug, positioning a sheath to deliver the fluid sealing plug through the sheath and into the opening through the biologic tissue membrane; and
inserting the fluid sealing plug and the support element into the sheath.

21. A method of closing an opening through a biologic tissue membrane and sealing against leakage, the method comprising:
inserting an implantable fluid sealing plug disposed on an elongated support element into the opening through a biologic tissue membrane, the sealing plug sealingly engaging the opening;
wherein the fluid sealing plug is compressible and absorbent.

22. The method of claim 21, further comprising:
prior to inserting the fluid sealing plug, positioning a sheath to deliver the fluid sealing plug through the sheath and into the opening through the biologic tissue membrane; and
inserting the fluid sealing plug and the support element into the sheath.
